(19) United States
(12) Patent Application Publication
(10) Pub. No.: US 2021/0364493 A1
(43) Pub. Date: Nov. 25, 2021

(54) APPARATUS FOR ANALYZING BODY FLUID AND METHOD FOR ANALYZING BODY FLUID USING THE SAME

(71) Applicant: NOUL CO., LTD., Yongin-si, Gyeonggi-do (KR)

(72) Inventors: Dong Young LEE, Yongin-si, Gyeonggi-do (KR); Eun Seong JEONG, Seocho-gu, Seoul (KR)

(21) Appl. No.: 17/057,280

(22) PCT Filed: Sep. 29, 2020
(86) PCT No.: PCT/KR2020/013368

§ 371 Date: Nov. 20, 2020

(30) Foreign Application Priority Data
Oct. 1, 2019 (KR) 10-2019-0121748

(51) Int. Cl. G01N 33/487; B41M 3/00; B41J 3/00; G01P 3/00; H04N 5/225

(52) U.S. Cl. CPC G01N 33/487; B41M 3/006; H04N 5/2256; G01P 3/00; B41J 3/00

(57) ABSTRACT

The present invention relates to a body fluid analyzing apparatus and a body fluid analyzing method using the same. This body fluid analyzing apparatus includes: an ejecting unit configured to eject a body fluid; a main body unit configured to support an ejecting unit; and a printing member on which the body fluid ejected through the ejecting unit is printed, wherein the body fluid is printed on a surface of the printing member while the printing member moves in one direction relative to the ejecting unit.

### TECHNICAL FIELD

The present invention relates to an apparatus for analyzing a body fluid and a method for analyzing a body fluid using the same, and to an apparatus for analyzing a body fluid and a method for analyzing a body fluid using the same, capable of rapidly and accurately printing a sufficient amount of the body fluid and improving the accuracy of examination by analyzing an image of the printed body fluid.

### BACKGROUND ART

A complete blood cell count CBC examination is a blood examination method for diagnosing the patient's health condition and diseases by identifying blood cells in blood, such as white blood cells, red blood cells, and platelets and performing counting and morphological analysis. A conventional CBC apparatus uses coulter or flow cytometry, which utilizes an indirect examination method for identifying and counting the blood cells by: allowing the blood to pass through a micro-fluidic channel, irradiating the same with laser, and measuring the split light; or applying current and measuring an amount of change in impedance. Such a CBC examination apparatus using the indirect examination method has relatively high accuracy for a normal blood sample, but there is a problem that it is difficult to accurately identify the type of blood cells in a blood sample in a disease state in which the blood cells, of which shape and size are changed due to immaturity, activation, or mutation, are present. Also, in a case where the platelets pass through the micro-fluidic channel in an agglomerate state, the platelets are counted as only one or identified as a whiter cell larger than the platelet, and thus, the reliability of data is deteriorated.

In the conventional CBC apparatus described above, analyzed information is compared with statistical data of a normal person, and a specific signal (flag signal) is transmitted to a sample showing an abnormal value, and in this case, experts make a diagnosis by manually smearing the sample, performing a visual examination through a microscope, and identifying abnormal blood cells. However, when the blood is directly smeared to identify abnormal blood cells from the obtained sample, the reliability of the examination data is deteriorated because the number of blood cells that can be smeared on the slide is limited to a specific area. Also, when the cells that are main indicators of certain diseases are, for example, circulating tumor cells (CTC) that move along the blood and cause metastasis of cancer, these cells are distributed in the blood at an extremely small percentage (about one of about 10⁶ to 10⁷ nucleated cells). Accordingly, to detect such cells, the experts have to repeat smear operations for a large number of samples by themselves, and thus there is a fundamental limitation in the number of samples that can be examined, and it is difficult to rapidly and accurately perform the examination.

Therefore, there is a need to develop an apparatus for analyzing a body fluid, which directly checks all blood cells in a sample to prevent an identification error in the indirect measurement method and has no limitation on the examination area so that all cells present in an extremely small amount in blood or other body fluids can be checked.

### DESCRIPTION OF EMBODIMENTS

### Technical Problem

Accordingly, the present invention has been made to solve the problem described above, and an object of the present invention is to provide an apparatus for analyzing a body fluid (hereinafter, referred to as a body fluid analyzing apparatus) and a method for analyzing a body fluid (hereinafter, referred to as a body fluid analyzing method) using the same, capable of improving the accuracy of examination by rapidly and accurately printing a sufficient amount of the body fluid and analyzing an image of the printed body fluid.

### Technical Solution to Problem

A body fluid analyzing apparatus according to one aspect of the present invention includes: an ejecting unit configured to eject a body fluid; a main body unit configured to support an ejecting unit; and a printing member on which the body fluid ejected through the ejecting unit is printed, wherein the body fluid is printed on a surface of the printing member while the printing member moves in one direction relative to the ejecting unit.

Also, the printing member, which is wound into a roll shape on a first rolling unit provided on one side, may be moved while being unwound from the first rolling unit and may be wound again into a roll shape on a second rolling unit provided on the other side, and

the body fluid ejected from the ejecting unit may be printed on the printing member when the printing member is moved to the second rolling unit while being unwound from the first rolling unit and is wound in a printed state on the second rolling unit.

Also, the printing member may be accommodated in a cartridge that is detachably coupled to the main body unit.

Also, a first rolling unit and a second rolling unit, which are coupled to respective ends of the printing member, may be provided in the cartridge, and

the printing member may be wound on the second rolling unit while being unwound from the first rolling unit, or may be wound on the first rolling unit while being unwound from the second rolling unit.

Also, the body fluid analyzing apparatus may include an optical unit configured to capture and analyze an image of the printed body fluid when the printing member on which the body fluid is printed moves.

Also, the printing member may be a film including a tape-type light transmitting material.

Also, the body fluid analyzing apparatus may include an optical unit which is provided in the main body unit to capture and analyze an image of the body fluid printed on the printing member, wherein, when the printing member is moved to the first rolling unit while being unwound from the second rolling unit and is wound again on the first rolling unit, the optical unit captures and analyzes the image of the body fluid printed on the printing member.

Also, the optical unit may include: a light source part which is coupled to the main body unit to provide a light source toward the printing member; an image capturing part which is positioned on the opposite side from the light source part to capture the image of the body fluid printed on the printing member; and an image analyzing part which uses the image captured by the image capturing part to perform image analysis.

Also, the body fluid analyzing apparatus may include: a first rotary shaft and a second rotary shaft which are provided in the main body unit and inserted into the first rolling unit and the second rolling unit, respectively; a first clutch selectively connected to the first rotary shaft; a second clutch selectively connected to the second rotary shaft; a power providing unit configured to transmit power to the first clutch or the second clutch; and a connection member configured to connect the first clutch and the second clutch and transmit the power, wherein the first clutch or the second clutch is selectively connected to the first rotary shaft or the second rotary shaft, respectively, by the power providing unit to selectively rotate the first rolling unit or the second rolling unit, thereby moving the printing member in one direction and winding the printing member.

Also, the body fluid analyzing apparatus may include: a first tension unit connected to a first connection shaft of the first clutch to maintain tension of the first rolling unit; and a second tension unit connected to a second connection shaft of the second clutch to maintain tension of the second rolling unit,

wherein, when the first rolling unit is connected to the first clutch and rotates, the second tension unit of the second rolling unit operates, and when the second rolling unit is connected to the second clutch and rotates, the first tension unit of the first rolling unit operates.

Also, the body fluid analyzing apparatus may include a speed measuring unit configured to sense a moving speed of the printing member when the printing member is unwound from the first rolling unit or the second rolling unit.

Also, the ejecting unit may include: a needle part configured to eject the body fluid; a mounting part to which the needle part is mounted; and a height adjusting part configured to space an end of the needle part from the printing member a predetermined distance.

Also, the height adjusting part may include a sensing unit configured to sense whether or not the end of the needle part comes into contact with the printing member when the mounting part approaches the printing member.

Also, the needle part may include a chamber made of a transmitting material so that the body fluid is visible from the outside.

Also, the body fluid analyzing apparatus may include a pressure supply unit which provides pressure so that the body fluid to be printed is suctioned into the ejecting unit or the body fluid is ejected from the ejecting unit.

Also, the pressure supply unit may include: a connection pipe connected to the ejecting unit; a conveyance pipe configured to convey oil from an oil tank which is filled with the oil; a branch pipe branched from an intersection point between the connection pipe and the conveyance pipe; a valve configured to selectively open and close the connection pipe, the conveyance pipe, and the branch pipe; a syringe pump which provides pressure so that the oil is suctioned through the conveyance pipe when the valve allows the conveyance pipe to communicate with the branch pipe, and the needle part suctions or ejects the body fluid when the valve allows the connection pipe to communicate with the branch pipe; and a control part configured to control an output of the syringe pump.

Also, the cartridge may include: a first exposure part which is exposed to the outside so that the body fluid is printed when the printing member is moved to the second rolling unit while being unwound from the first rolling unit; and a second exposure part which is exposed to the outside so that an image of the printed body fluid is captured when the printing member wound on the second rolling unit is moved to the first rolling unit while being unwound from the second rolling unit again, wherein a first insertion hole, into which a light source part that provides a light source to capture the image of the printed body fluid is inserted, is provided below the second exposure part.

Also, the body fluid may include blood, lymph, tissue fluid, cerebrospinal fluid, in-vitro culture, chopped tissue, tears, amniotic fluid, semen, or urine.

Also, the optical unit may analyze at least one of blood cells, cancer cells, endothelial cells, epithelial cells, bacteria, parasites, cultured cells, and exosomes, which are contained in the body fluid.

Also, the body fluid analyzing apparatus may include: a staining unit configured to selectively stain cells of the printed body fluid; and an optical unit configured to capture and analyze an image of the body fluid having the stained cells.

A body fluid analyzing method according to another aspect of the present invention includes: an ejecting step of ejecting body fluid from an ejecting unit; a printing step of, while moving a printing member in one direction, printing the body fluid on a surface of the printing member when the body fluid is ejected by the ejecting step; and an image capturing and analyzing step of capturing an image of the body fluid printed by the printing step and analyzing the body fluid.

Here, the printing member may include a tape-type film, wherein the printing member, which is wound into a roll shape on a first rolling unit provided on one side, is moved while being unwound from the first rolling unit and is wound into a roll shape on a second rolling unit provided on the other side, and the printing member, which is wound on the second rolling unit, is moved while being unwound from the second rolling unit and is wound again on the first rolling unit,

wherein the printing step is performed when the printing member is moved to the second rolling unit while being unwound from the first rolling unit, and

the image capturing and analyzing step is performed when the printing member is moved to the first rolling unit while being unwound from the second rolling unit and is wound again on the first rolling unit.

Here, the body fluid may include blood, lymph, tissue fluid, cerebrospinal fluid, in-vitro culture, chopped tissue, tears, amniotic fluid, semen, or urine.

Here, in the image capturing and analyzing step, at least one of blood cells, cancer cells, endothelial cells, epithelial cells, bacteria, parasites, cultured cells, and exosomes, which are contained in the body fluid, may be analyzed.

Here, the body fluid analyzing method may include a staining step of selectively staining cells of the printed body fluid, wherein, in the image capturing and analyzing step, an image of the body fluid having the printed cells is captured and analyzed.

### Advantages Effects of Disclosure

In the body fluid analyzing apparatus according to the present invention and the body fluid analyzing method using the same, all the individual cells such as the blood cells contained in samples may be directly checked by printing all the samples of the sufficient amount of body fluid as a monolayer and imaging the same. Accordingly, since the identification error may be prevented, and there is no limitation on the examination area, all the cells present in the extremely small amount in the body fluid such as the blood may be examined and checked thoroughly. Therefore, the present invention may significantly improve the reliability of cell count data.

Also, the present invention may be widely applied to the analysis for the cells contained in various body fluids including, for example, blood, lymph, tissue fluid, cerebrospinal fluid, in-vitro culture, urine, and the like, which are the various body fluids obtained from the subject.

Also, according to the present invention, since the body fluid is printed on the plane that moves in one direction, a large amount of body fluid may be easily printed, and the image analysis thereof may be easily performed.

Also, according to the present invention, since the body fluid is printed while the printing member accommodated in the cartridge is moved in one direction, the body fluid to be examined may be significantly increased. Also, since the image of the printed body fluid is captured and analyzed while the printing member on which the body fluid is printed is moved in the other direction, the printing, imaging capturing, and analysis may be effectively performed even in a small space.

Also, according to the present invention, since the collection and ejection of the body fluid sample are controlled by a pressing method using oil, the spraying and precise printing are possible at a constant volume.

### BRIEF DESCRIPTION OF DRAWINGS

FIG. 1 is a perspective view of a body fluid analyzing apparatus according to an embodiment of the present invention.

FIG. 2 is a view showing a state in which a cartridge of FIG. 1 is separated.

FIG. 3 is an enlarged view of an ejecting unit.

FIG. 4 is an exploded perspective view of the cartridge.

FIG. 5 is a perspective view, taken from a different angle with respect to FIG. 1.

FIG. 6 is a plan view of FIG. 1.

FIG. 7 is a view illustrating extracted main parts of FIG. 6.

FIG. 8 is a perspective view of a pressure supply unit.

FIG. 9 is a block diagram of the pressure supply unit.

FIG. 10(a) and FIG. 10(b) are schematic views of a state in which an image is captured by an optical unit.

FIG. 11 is a flowchart in which the captured image is analyzed by the optical unit.

FIG. 12 is a block diagram of a body fluid analyzing apparatus in which a staining unit is added.

FIG. 13 is a flowchart of a body fluid analyzing method according to an embodiment of the present invention.

FIG. 14 is a flowchart of a body fluid analyzing method according to another embodiment of the present invention.

### MODE OF DISCLOSURE

Hereinafter, various embodiments of the present invention will be described in association with the accompanying drawings. Since the present invention may have diverse modifications and various embodiments, specific embodiments are illustrated in the drawings, and their detailed descriptions are given. However, this is not intended to limit the various embodiment of the present invention within specific embodiments, and it should be understood that the present invention covers all the modifications and/or replacements within the idea and technical scope of the various embodiments of the present invention. In the description of drawings, like reference numerals are used for referring to similar elements.

The expression of “comprise” or “may comprise”, which can be used in the various embodiments of the present invention, indicates the presence of the relevant functions, operations, or elements in the disclosure and does not limit one or more additional functions, operations, or elements. Also, in the various embodiments of the present invention, it will be understood that the term “includes” or “comprises”, when used in this specification, specifies the presence of stated features, numbers, steps, operations, elements, components, or a combination thereof, but does not preclude the presence or addition of one or more other features, numbers, steps, operations, elements, components, or combinations thereof.

Also, term “ . . . part” represents a unit that processes at least one function or operation, and this may be embodied as hardware or software or embodied as a combination of hardware and software.

It will be understood that when an element is referred to as being “connected to” another element, the element can be directly connected to another element, or there may be an intervening element between the element and another element. On the other hand, it will be understood that when an element is referred to as being “directly connected to” or “directly coupled to” another element, there is no intervening element between the element and another element.

The terms used in the various embodiments of the present invention are used only for explaining a specific embodiment and not intended to limit to the various embodiments of the present invention. The singular forms include the plural forms unless the context clearly indicates otherwise.

Unless otherwise defined, all terms used herein including technical or scientific terms have the same meanings as those generally understood by a person with ordinary skill in the art to which the various embodiments of the present invention pertains.

Terms as defined in a commonly used dictionary should be construed as having the same meaning as in an associated technical context, and unless defined apparently in the various embodiments of the present invention, are not to be understood abnormally or as having an excessively formal meaning.

[symbol]

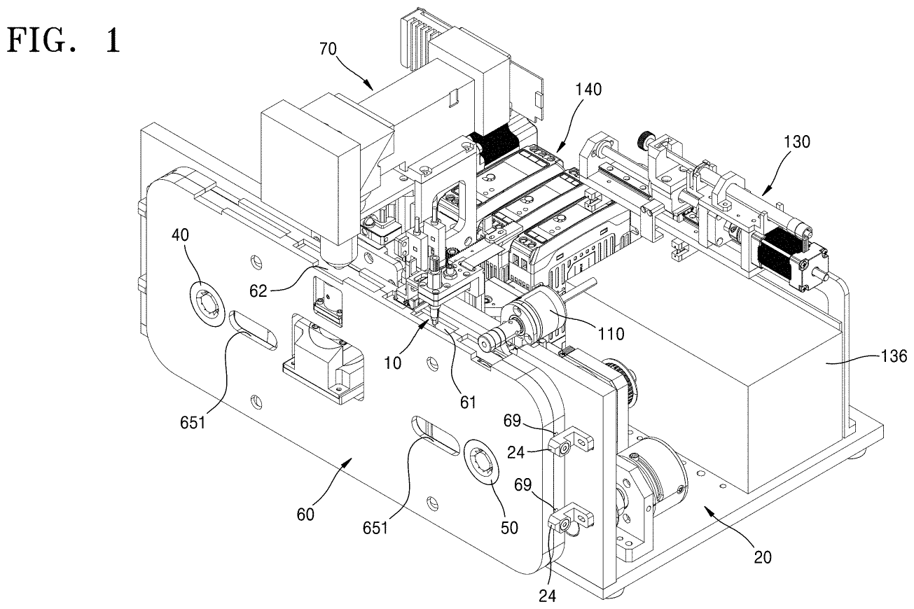

Hereinafter, exemplary embodiments according to the present invention will be described in detail with reference to the accompanying drawings. Referring to FIG. 1, a body fluid analyzing apparatus according to an embodiment of the present invention includes an ejecting unit 10, a main body unit 20, and a printing member 30.

[symbol]

First of all, the body fluid analyzing apparatus according to the present invention and a body fluid analyzing method including the same may improve the accuracy of examination by rapidly printing a large amount of a body fluid, creating an image of the body fluid, and inspecting the image. In the present invention, the body fluid is a biological sample separated from a subject, and the subject includes a human and an animal. Particularly, the body fluid includes blood, lymph, tissue fluid, cerebrospinal fluid, in-vitro culture, chopped tissue, tears, amniotic fluid, semen, urine, or the like. That is, the body fluid analyzing apparatus and the body fluid analyzing method using the same according to the present invention are involved in both a body fluid obtained directly from the subject and a body fluid subjected to secondary treatment such as mixing a body fluid obtained from the subject with a solution and the like. Also, the body fluid analyzing apparatus and the body fluid analyzing method using the same according to the present invention may perform examinations and analysis for various detection targets such has blood cells, cancer cells, endothelial cells, epithelial cells, bacteria, parasites, cultured cells, and exosomes, which are contained in the body fluid.

[symbol]

Ejecting Unit 10

[symbol]

The ejecting unit 10 is provided to eject a body fluid to be examined, and the main body unit 20 supports the ejecting unit 10. The printing member 30 is provided to print the body fluid which is to be ejected through the ejecting unit 10. According to the embodiment, the body fluid is printed on a surface of the printing member 30 while the printing member 30 is moved relative to the ejecting unit 10 in one direction. According to the embodiment, the ejecting unit 10 is fixed to the main body unit 20, and the printing member 30 is moved relative to the ejecting unit 10. Alternatively, the ejecting unit 10 may be embodied as moving relative to the printing member 30.

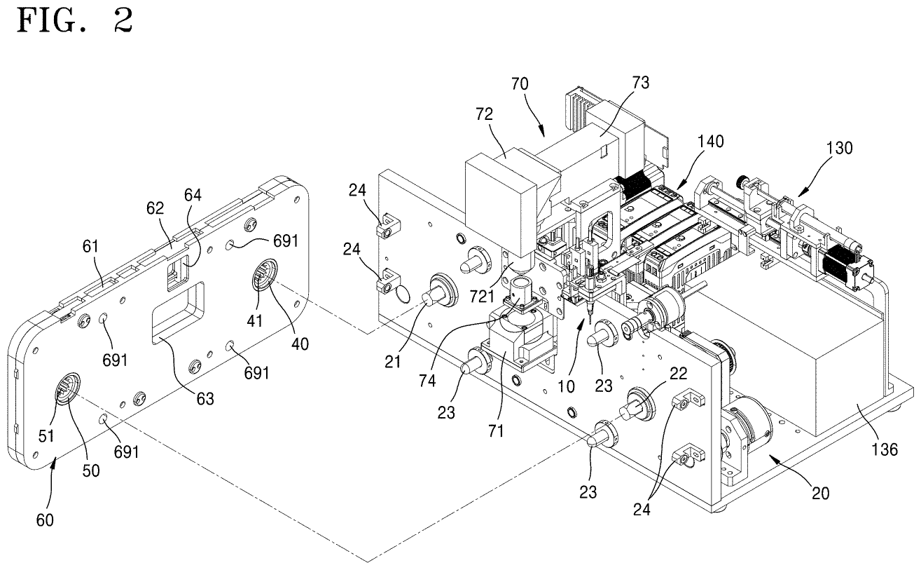

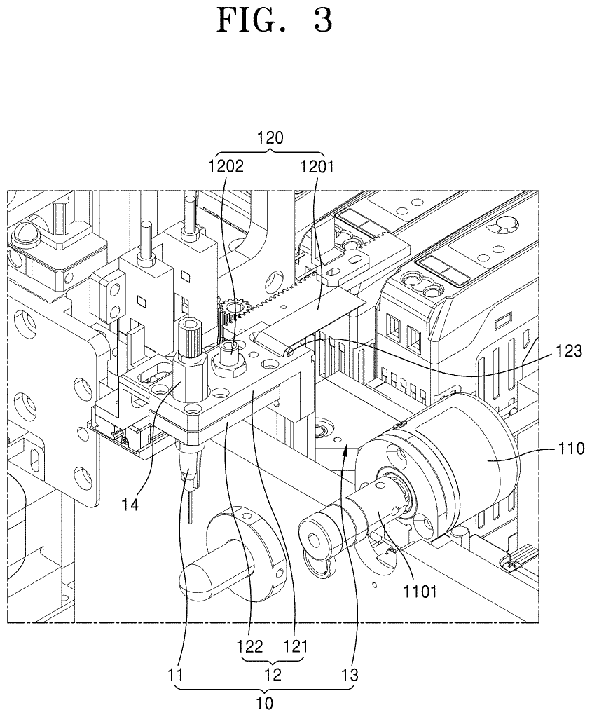

Referring to FIGS. 2 and 3, the ejecting unit 10 includes a needle part 11, a mounting part 12, and a height adjusting part 13.

The needle part 11 is provided to accommodate a body fluid, which is to be examined, and eject the body fluid onto the printing member 30. According to the embodiment, the needle part 11 includes a chamber 14 including a light transmitting material so that the body fluid is visible from the outside.

The mounting part 12 is provided so that the needle part 11 is mounted thereto. According to the embodiment, an upper plate 121 and a lower plate 122 of the mounting part 12 are coupled while overlapping each other in parallel, and a mounting hole, to which the needle part 11 is mounted, is provided in each of the upper plate 121 and the lower plate 122.

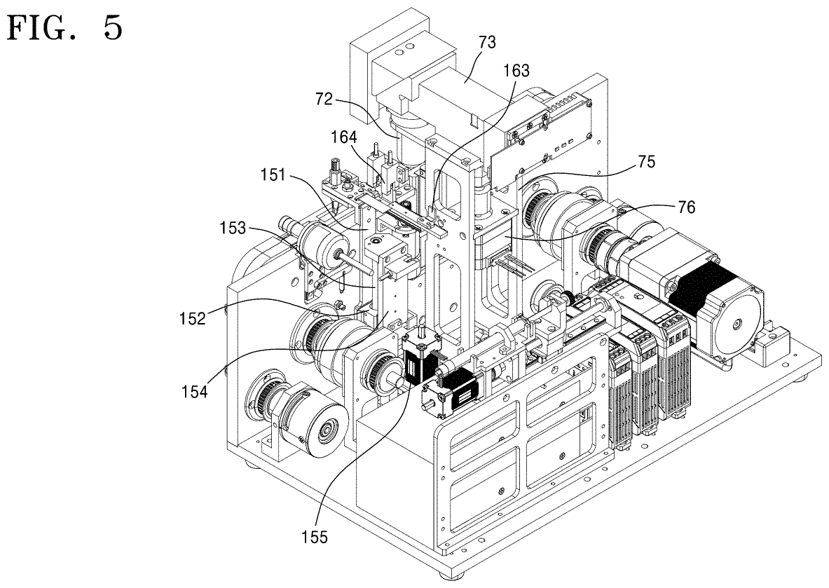

The height adjusting part 13 is provided to space an end of the needle part 11 from the printing member 30 a predetermined distance. As illustrated in FIG. 5, the height adjusting part 13 includes a support block 151 for supporting the mounting part 12, a moving block 152 coupled to the support block 151 and ascending and descending together with the support block 151, a first screw shaft 153 coupled to the mounting block 152 and disposed vertically, a support part 154 for supporting the first screw shaft 153, and a first step motor 155 for providing a rotation force to the first screw shaft 153 so that the moving block 152 moves vertically along the first screw shaft 153. The moving block 152 is raised and lowered by the first step motor 155, and consequently, the needle part 11 mounted to the mounting part 12 gets closer to or moves away from the printing member 30.

The embodiment includes a sensing unit 120 for sensing whether or not the end of the needle part 11 comes into contact with the printing member 30.

Referring to FIG. 3, the sensing unit 120 includes a film-type pressure sensor 1201 which is disposed between the upper plate 121 and the lower plate 122 and a pin part 1202 which transmits, to the pressure sensor 1201, the pressure when the needle part 11 comes into contact with the printing member 30. The pin part 1202 passes through the upper plate 121 and is installed such that a lower end thereof comes into contact with a surface of the pressure sensor 120. An exposure hole 123 is provided in the mounting part 12 to connect the film-type pressure sensor 1201 to an external terminal.

When the needle part 11 is lowered by the height adjusting part 13 and brought into contact with the printing member 30, the end of the needle part 11 collides with the printing member 30 to generate an impact force, and the pressure due to the impact is transmitted to the film-type pressure sensor 1201 by the pin part 1202. That is, the pin part 1202 descends together as the mounting part 12 descends, and when the needle part 11 collides with the printing member 30, the pin part 1202 impacts the pressure sensor 1201 due to inertia. When the impact is sensed by the pressure sensor 1201, the pressure sensor 1201 transmits an impact sensing signal to a control part 136. When receiving the impact sensing signal, the control part 136 allows the height adjusting part 13 to raise the mounting part 12 so that a predetermined distance is maintained between the end of the needle part 11 and the printing member 30. Thus, the end of the needle part 11 is spaced a predetermined distance from the printing member 30. According to the embodiment, the control part 136 controls not only the ejecting unit 10 but also movement of an optical unit 70, a power providing unit 80, a pressure supply unit 130, a loading block 160, and the like.

According to the embodiment, the first step motor 155 vertically moves the moving block 152 while rotating the first screw shaft 153 precisely machined, and one rotation of the screw may move the mounting part 12 by 0.25 mm. When the body fluid is blood, the needle part 11 may be slightly spaced apart from the printing member 30 by a range of 10 to 15 μm.

[symbol]

Main Body Unit 20

[symbol]

The mounting part 20 is provided to support the ejecting unit 10. According to the embodiment, other components constituting the body fluid analyzing apparatus according to the embodiment are coupled and disposed in the main body unit 20. For example, in the main body unit 20, there are disposed components such as the ejecting unit 10, a cartridge 60 for accommodating the printing member 30, the power providing unit 80 for providing power and peripheral components thereof to move the printing member 30 in a winding or unwinding direction, the optical unit 70, a power supply unit 140 for supplying electric power, and the pressure supply unit 130. As illustrated in FIGS. 1 and 2, etc., the main body unit 20 is illustrated such that the inside thereof is visible for convenience.

[symbol]

Printing Member 30

[symbol]

The printing member 30 is provided to print the body fluid which is to be ejected through the ejecting unit 10. According to the embodiment, the printing member 30 includes a film of a tape-type light transmitting material. The printing member 30 includes a hydrophilic material, and thus, the body fluid may be easily printed on one surface of the film. According to the embodiment, the printing member 30 includes the transparent film, but according to another embodiment, the printing member 30 may include a translucent film through which a light source of the optical unit 70 may pass. The body fluid ejected from the ejecting unit 10 is printed as a single layer on the printing member 30. To examine and analyze all body fluids to be examined, detection targets such as blood cells contained in the body fluid are printed as a single layer on the printing member 30 that provides a single plane. When the body fluid is printed as a single layer, an image thereof may be easily created, and all the body fluids to be examined may be examined thoroughly.

According to the embodiment, the printing member 30, which is wound into a roll shape on a first rolling unit 40 provided on one side, is moved while being unwound from the first rolling unit 40 and is wound again into a roll shape on a second rolling unit 50 provided on the other side. The body fluid ejected from the ejecting unit 10 is printed on the printing member 30 when the printing member 30 is moved to the second rolling unit 50 while being unwound from the first rolling unit 40, and is wound in a printed state on the second rolling unit 50. Since the printing member 30 is wound on the first and second rolling units 40 and 50, a long printable area may be secured.

[symbol]

Cartridge 60

[symbol]

According to the embodiment of the present invention, the printing member 30 is accommodated in the cartridge 60 detachably coupled to the main body unit 20, and the first rolling unit 40 and the second rolling unit 50 are provided within the cartridge 60. However, the first rolling unit 40 and the second rolling unit 50 are not limited as being provided within the cartridge 60. For example, the first rolling unit 40 and the second rolling unit 50 may be provided separate therefrom. In this case, the printing member 30 wound on the first rolling unit 40 may be unwound therefrom and wound again on the second rolling unit 50, and the ejecting unit 10 may perform printing of the body fluid while the printing member 30 moves from the first rolling unit 40 to the second rolling unit 50.

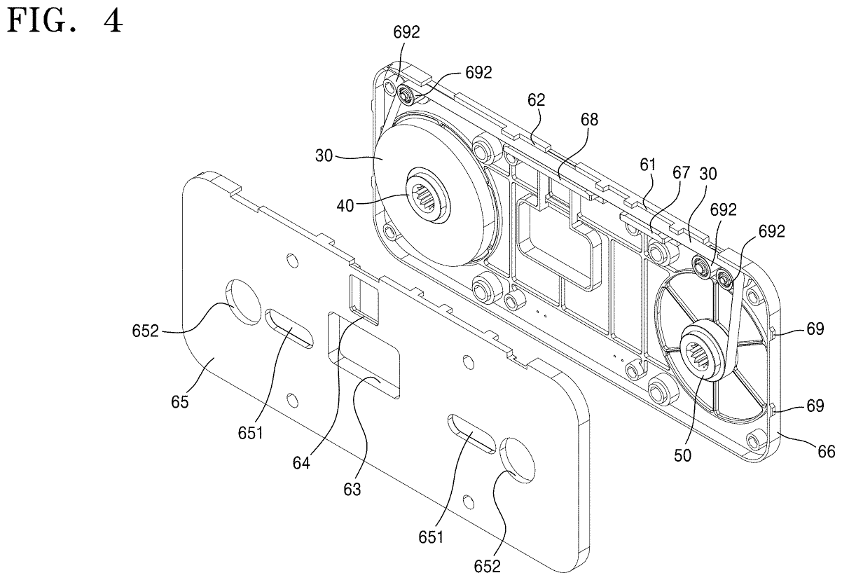

As illustrated in FIGS. 2 and 4, the cartridge 60 is provided with the first rolling unit 40 and the second rolling unit 50, which are disposed spaced apart from each other. The respective ends of the printing member 30 are coupled to the first rolling unit 40 and the second rolling unit 50. The printing member 30 may be wound on the second rolling unit 50 while being unwound from the first rolling unit 40, or may be wound on the first rolling unit 40 while being unwound from the second rolling unit 50. That is, while the first rolling unit 40 and the second rolling unit 50 rotate in one direction, the printing member 30, which is wound on the first rolling unit 40, is moved to the second rolling unit 50 and wound thereon. Also, while the first rolling unit 40 and the second rolling unit 50 rotate in the other direction opposite to the one direction, the printing member 30, which is wound on the second rolling unit 50, is moved to the first rolling unit 40 and wound thereon.

According to the embodiment, the cartridge 60 is provided with a front part 65 and a rear part 66, which are detachable from each other. Coupling holes 652, into which the first rolling unit 40 and the second rolling unit 50 are inserted, are provided in the front part 65. Also, in the vicinity of the coupling holes 652 in the front part 65, there are provided elongated holes 651 which check an amount of the printing member 30 wound on the first rolling unit 40 and the second rolling unit 50. In the rear part 66, there are provided the first rolling unit 40, the second rolling unit 50, and a plurality of guide rollers 692 for guiding the printing member 30.

A first rotary shaft 21 and a second rotary shaft 22, which are provided in the main body unit 20, are inserted into the first rolling unit 40 and the second rolling unit 50, respectively. A first hook part 41, which is saw-toothed to prevent slip when the first rotary shaft 21 rotates, is provided in the first rolling unit 40, and a second hook part 51, which is saw-toothed to prevent slip when the second rotary shaft 22 rotates, is provided in the second rolling unit 50.

The cartridge 60 employed in the embodiment will be further described. A first exposure part 61, a second exposure part 62, and a first insertion hole 63 are provided in the cartridge 60.

The first exposure part 61 is a region exposed to the outside so that the body fluid is printed when the printing member 30 is moved to the second rolling unit 50 while being unwound from the first rolling unit 40. A first support piece 67 for supporting the printing member 30 is provided below the first exposure part 61.

The second exposure part 62 is a region for exposing the printing member 30 to the outside so that an image of the printed body fluid may be captured when the printing member 30, which is wound on the second rolling unit 50, is unwound therefrom again and moved to the first rolling unit 40. A second support piece 68 for supporting the printing member 30 is provided below the second exposure part 62.

The first insertion hole 63 is provided below the second exposure part 62 so that a light source part 71, which provides a light source to capture the image of the printed body fluid, may be inserted thereinto. The light source part 71 will be described later in detail.

[symbol]

Optical Unit 70

[symbol]

The optical unit 70 is provided to capture and analyze an image of the printed body fluid when the printing member 30, on which the body fluid is printed, moves. The ejecting unit 10 prints the body fluid on a top surface of the printing member 30 when the printing member 30 moves, and the optical unit 70 captures and analyze the image of the printed body fluid. Therefore, a large amount of the body fluid is rapidly printed to obtain images thereof, and this has an effect similar to directly observing all body fluids to be examined and significantly improves the accuracy of examination.

After the printing of the body fluid on the printing member 30 is finished, the optical unit 70 utilized in the embodiment captures and analyzes the image of the body fluid printed on the printing member 30 when the printing member 30 is wound again on the first rolling unit 40 while being unwound from the second rolling unit 50. That is, according to the embodiment of the present invention, the printing member 30 accommodated in the cartridge 60 may be moved in both directions while being wound or unwound. Here, when moved in one direction, the printing is performed, and when moved in the other direction, the optical analysis is performed.

Obviously, since the main function of the optical unit 70 is to capture and obtain the image of the body fluid printed on the printing member 30, the optical unit 70 is not limited to only the case in which the printing member 30 is accommodated in the cartridge 60 and moved in both directions. For example, in a case where the printing member 30 wound on the first rolling unit 40 is only moved to the second rolling unit 50 (not wound again on the first rolling unit 40), the optical unit 70 is disposed on a rear side of the ejecting unit 10. Thus, before wound on the second rolling unit 50, the printing is performed by the ejecting unit 10, and the image may be obtained by the optical unit 70.

According to the embodiment, the optical unit 70 includes a light source part 71, an image capturing part 72, and an image analyzing part 73.

The light source part 71 is provided to supply a light source toward the printing member 30. The light source part 71 is coupled to the main body unit 20. In particular, the light source part 71 is coupled to a side wall of the main body unit 20 while protruding toward the outside. When the cartridge 60 is coupled to the main body unit 20, the light source part 71 is coupled to a first insertion hole 63 of the cartridge 60 in an inserted state.

According to the embodiment, the light source part 71 emits light toward a bottom surface of the printing member 30. A light collecting part 74 is provided above the light source part 71 so that the light is collected to the printing member 30. According to the embodiment, the light collecting part 74 has a cylindrical shape, and similar to the light collecting part 71, the light collecting part 74 protrudes outward from the side wall of the main body unit 20. Also, a second insertion hole 64, through which the light collecting part 74 is inserted, is provided in the cartridge 60.

The image capturing part 72 is positioned on the opposite side from the light source part 71 and captures an image of the body fluid printed on the printing member 30. The image capturing part 72 is disposed above the printing member 30, and the light source part 71 is disposed below the printing member 30. According to the embodiment, since the printing member 30 includes the transparent film, the light emitted from the light source part 71 passes through the body fluid printed on the printing member 30 and then enters the image capturing part 72. In another embodiment, even in a case where the printing member 30 includes the translucent film, the light emitted from the light source part 71 may pass through the printing member 30 and then enter the image capturing part 72.

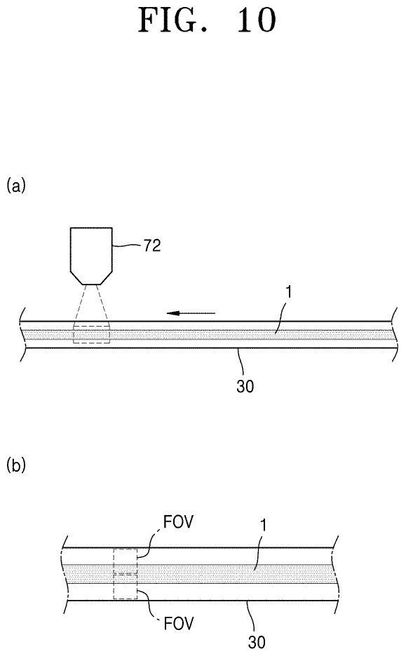

As illustrated in FIG. 10, the image capturing part 72 continuously captures a plurality of times images of the body fluid printed on the printing member 30 while the printing member 30 moves. The image capturing part 72 includes a lens part 721, and in a case where the lens part 721 having 20-50× magnification is used, the body fluid is printed with a width corresponding to a field of view (FOV) of the lens part 721. Also, in a case where the body fluid is printed with a width greater than the FOV of the lens part, the image capturing part 72 may capture images a plurality of times and secure the images of the body fluid over all the areas therebelow as illustrated in FIG. 10(b).

The image analyzing part 73 is provided to perform the image analysis by using the image captured by the image capturing part 72. The image analyzing part 73 analyzes each of a plurality of images captured by the image capturing part 72. The image analyzing part 73 has basic information about sizes, types, and shapes of normal blood cells and determines whether or not all the blood cells captured in each of the images are normal. Through such a process, there is an effect of examining all body fluids that have a large number of samples. The image analyzing part 73 consistently accumulates and manages data of the analyzed images and uses a deep learning program in which the data is utilized again to analyze new samples. Thus, whether an individual blood cell is abnormal may be rapidly and reliably determined.

[symbol]

Parts for Driving Cartridge 60

[symbol]

An embodiment of the present invention includes a first clutch 81, a second clutch 82, a power providing unit 80, and a connection member 83, to move the printing member 30 wound on the cartridge 60 in one direction or the other direction opposite thereto.

First, in the main body unit 20, there are provided a first rotary shaft 21 and a second rotary shaft 22, which are inserted into the first rolling unit 40 and the second rolling unit 50, respectively. According to the embodiment, the first rotary shaft 21 and the second rotary shaft 22 are inserted into the first rolling unit 40 and the second rolling unit 50 of the cartridge 60, respectively. Each of ends of the first rotary shaft 21 and the second rotary shaft 22 has a prism shape. Obviously, the shapes of the first and second shafts 21 and 22 are not limited thereto. The first rotary shaft 21 and the second rotary shaft 22 are inserted into the first rolling unit 40 and the second rolling unit 50, respectively, and are locked by a first hook part 41 and a second hook part 51, which are saw-toothed and provided on inner circumferential surfaces of the first rolling unit 40 and the second rolling unit 50, respectively. Accordingly, slipping is prevented during the rotation.

A coupling protrusion 69 is provided on an outer circumferential surface of the cartridge 60, and a mounting bracket 24 is provided in the main body unit 20 so that the coupling protrusion 69 is locked to prevent the cartridge 60 from being separated. Also, to stably couple the cartridge 60 to the main body unit 20, a plurality of coupling pins 23 are provided in the main body unit 20, and pin holes 691, into which the coupling pins 23 are inserted, are provided in the cartridge 60. As described above, when the cartridge 60 is mounted to the main body unit 20, the power providing unit 80 supplies power for moving the printing member 30 for the printing of the body fluid and the image capturing by the optical unit 70.

The first clutch 81 is selectively connected to the first rotary shaft 21. The second clutch 82 is selectively connected to the second rotary shaft 22. In the present specification, “being selectively connected to” represents that the first clutch 81 or the second clutch 82 may be connected or not connected to the first rotary shaft 21 or the second rotary shaft 22.

The power providing unit 80 transmits the power to the first clutch 81 or the second clutch 82. The connection member 83 connects the first clutch 81 to the second clutch 82 and transmits the power. According to the embodiment, the power providing unit 80 serves as a motor to transmit the rotation force to the first rotary shaft 21 or the second rotary shaft 22. The first clutch 81 or the second clutch 82 is selectively connected to the first rotary shaft 21 or the second rotary shaft by the power providing unit 80, and the first rolling unit 40 or the second rolling unit 50 is selectively rotated to move and wind the printing member 30 in one direction.

In more detail, when the printing member 30 moves from the first rolling unit 40 to the second rolling unit 50, the second rolling unit 50 pulls the printing member 30 while rotating. To this end, the first clutch 81 is disconnected from the first rotary shaft 21, and the second clutch 82 is connected to the second rotary shaft 22. According to the embodiment, since the first clutch 81 and the second clutch 82 are connected to each other through the connection member 83, the rotation force from the power providing unit 80 is transmitted to the second clutch 82 through the connection member 83, and consequently, the second rotary shaft 22 rotates. On the other hand, since the first rotary shaft 21 is not connected to the first clutch 81, the first rotary shaft 21 is rotated by the force when the second rolling unit 50 pulls the printing member 30 while rotating, and the printing member 30 is unwound.

On the other hand, when the printing member 30 moves from the second rolling unit 50 to the first rolling unit 40, the connections of the first and second clutches are reversed. That is, the first clutch 81 is connected to the first rotary shaft 21 and thus transmits the power of the power providing unit 80, and the second clutch 82 is disconnected from the second rotary shaft 22. Here, the connection member 83 rotates in a state in which the first clutch 81 and the second clutch 82 are connected to each other, but the second clutch 82 is disconnected from the second rotary shaft 22. Thus, the force is not transmitted to the second rotary shaft 22. The first rotary shaft 21 rotates, and accordingly, the first rolling unit 40 rotates to pull the printing member 30. The second rotary shaft 22 is rotated by the force with which the first rolling unit 40 pulls the printing member 30, and the printing member 30 is unwound.

According to an embodiment of the present invention, there are provided a first tension unit 90 and a second tension unit 100 for maintaining tension of the printing member 30 when the printing member 30 is selectively pulled by the first rolling unit 40 or the second rolling unit 50.

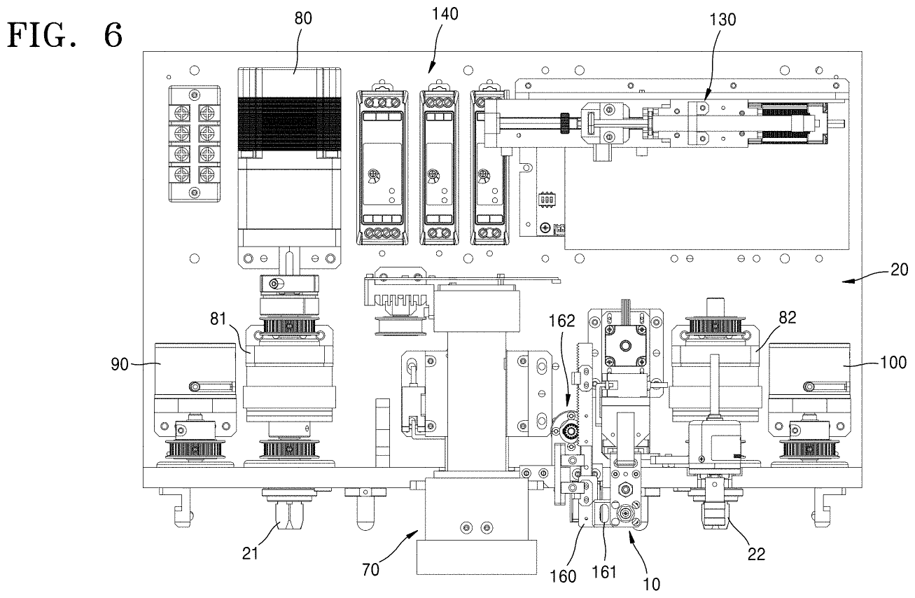

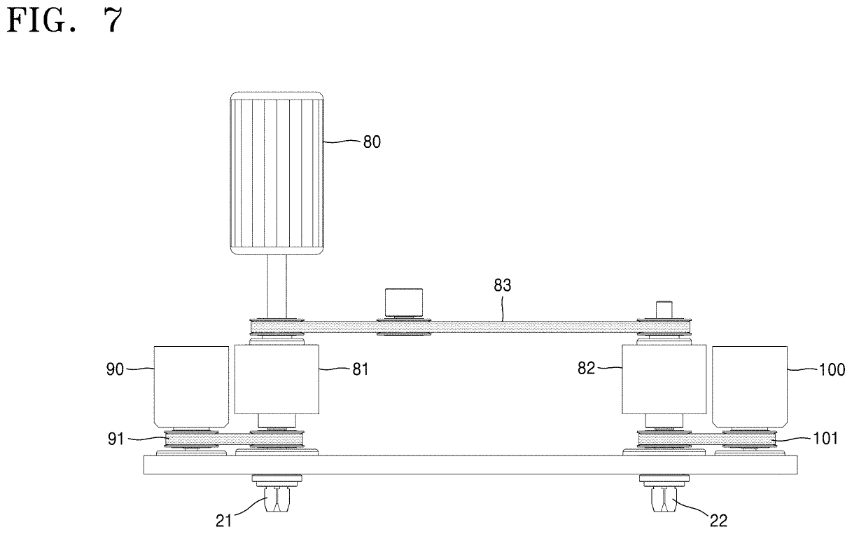

As illustrated in FIGS. 6 and 7, the first tension unit 90 is connected to a first connection shaft of the first clutch 81 to maintain the tension in the first rolling unit 40. The first tension unit 90 and the first connection shaft are connected to each other by a first belt 91. The first tension unit 90 operates when the second rolling unit 50 pulls the printing member 30 while being connected to the second clutch 82 and rotating. The first belt 91 allows the first rolling unit 40 to be rotatable under appropriate tension.

The second tension unit 100 is connected to a second connection shaft of the second clutch 82 to maintain the tension in the second rolling unit 50. The second tension unit 100 and the second connection shaft are connected to each other by a second belt 101. The second tension unit 100 operates when the first rolling unit 40 pulls the printing member 30 while being connected to the first clutch 81 and rotating. The second belt 101 allows the second rolling unit 50 to be rotatable under appropriate tension.

According to the embodiment, there is provided a speed measuring unit 110 which senses the moving speed of the printing member 30 when the printing member 30 is unwound from the first rolling unit 40 or the second rolling unit 50. According to the embodiment, the speed measuring unit 110 is an encoder having a cylindrical shaft 1101 that rotates in contact with the printing member 30. When the printing member 30 moves, the cylindrical shaft 1101 measures the moving speed of the printing member 30 while rotating. The control part 136 controls the rotation force transmitted by the power providing unit 80 so that the moving speed of the printing member 30 is maintained at an appropriate speed, and according to the controlled speed, the printing member 30 is controlled to move under appropriate tension by the first tension unit 90 and the second tension unit 100.

[symbol]

Pressure Supply Unit 130 for Finely Regulating an Amount of Body Fluid

[symbol]

According to the embodiment of the present invention, provided is a pressure supply unit 130 for allowing a small amount of body fluid to be precisely ejected through the ejecting unit 10.

The pressure supply unit 130 supplies pressure so that the body fluid to be printed on the printing member 30 is suctioned into the ejecting unit 10, or the body fluid is ejected through the ejecting unit 10. According to the embodiment, a body fluid sample to be examined may be placed in a slot 161 provided in a loading block 160. The loading block 160 may be moved forward and backward by a loading block driving part 162. According to the embodiment, the loading block driving part 162 includes a motor and a rack and pinion structure. Also, there are provided a first object detecting sensor 164 and a first probe 163 for limiting a moving distance when the loading block 160 moves forward. The first probe 163 is provided in the loading block 160, and when the loading block moves forward and the first probe 163 is sensed by the first object detecting sensor 164, the movement of the loading block 160 is controlled to be limited.

When the loading block 160 moves forward, the body fluid sample is placed in the slot 161 provided in the loading block 160. Here, the ejecting unit 10 moves upward. When the loading block 160 moves backward and moves below the ejecting unit 10, the ejecting unit 10 descends and suctions the body fluid sample. Here, the pressure supply unit 130 supplies the pressure so that the body fluid is suctioned into the ejecting unit 10. The loading of the body fluid is finished when a predetermined amount of the body fluid is accommodated in the ejecting unit 10. Also, when the body fluid is printed on the printing member 30 by the ejecting unit 10, the pressure supply unit 130 supplied the pressure so that the body fluid is ejected from the ejecting unit 10.

In particular, according to the embodiment, the pressure supply unit 130 includes a connection pipe 131, a conveyance pipe 132, a branch pipe 133, a valve 134, a syringe pump 135, and a control part 136.

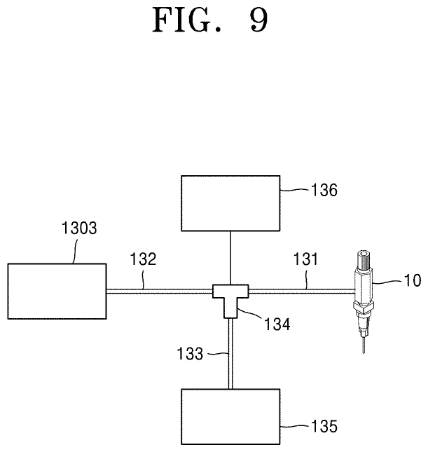

As illustrated in FIG. 9, the connection pipe 131 is connected to the ejecting unit 10, and the conveyance pipe 132 extends from an oil tank 1303 filled with oil so that the oil is conveyed from the oil tank 1303. The branch pipe 133 is branched from an intersection point between the connection pipe 131 and the conveyance pipe 132. The valve 134 is provided to selectively open and close the connection pipe 131, the conveyance pipe 132, and the branch pipe 133. The connection pipe 131, the conveyance pipe 132, and the branch pipe 133 are connected to the one valve 134. The valve 134 allows the conveyance pipe 132 and the branch pipe 133 to communicate with each other but closes the connection pipe 131, or allows the branch pipe 133 and the connection pipe 131 to communicate with each other but closes the conveyance pipe 132.

The syringe pump 135 provides pressure so that the oil may be suctioned through the conveyance pipe 132 when the valve 134 allows the conveyance pipe 132 to communicate with the branch pipe 133, and the needle part 11 of the ejecting unit 10 suctions or ejects the body fluid when the valve 134 allows the connection pipe 131 to communicate with the branch pipe 133. That is, according to the embodiment, the body fluid to be loaded to the ejecting unit 10 is accommodated in the chamber 14, the needle part 11 and the syringe pump 135 are connected to each other by the connection pipe 131, and the syringe pump 135 and the oil tank 1303 are connected to the conveyance pipe 132. The conveyance pipe 132 and the connection pipe 131 are filled with the oil supplied from the oil tank 1303.

The control part 136 controls the syringe pump 135, and accordingly, the suction amount of the body fluid to be loaded through the ejecting unit 10 or the ejection amount of the body fluid to be ejected through the ejecting unit 10 are regulated. When the body fluid is suctioned for the loading of the body fluid, the control part 136 allows the syringe pump 135 to suction the oil of the connection pipe 131, and accordingly, the body fluid is suctioned through an end of the needle part 11. Also, when the body fluid is ejected for the printing, the control part 136 allows the syringe pump 135 to push the oil of the connection pipe 131 toward the needle part 11. Accordingly, the oil pushes the body fluid, and the body fluid is discharged.

As described above, the body fluid is indirectly suctioned or ejected by using the oil through a medium. Since the syringe pump 135 suctions or ejects the body fluid using the oil through a medium, the control part 136 may control precisely and quantitatively the suction amount and the ejection amount of the body fluid, compared to providing the suction force or the ejection pressure directly to the body fluid.

Also, the body fluid is not mixed with the oil, and when the examination of one sample is finished, the body fluid remaining in the connection pipe 131 is ejected together with a predetermined oil by the syringe pump 135, and the body fluid remaining in the ejecting unit 10 is removed. Also, the ejected oil is supplemented from the oil tank 1303, and accordingly, the ejecting unit 10 may be hygienically managed without contamination.

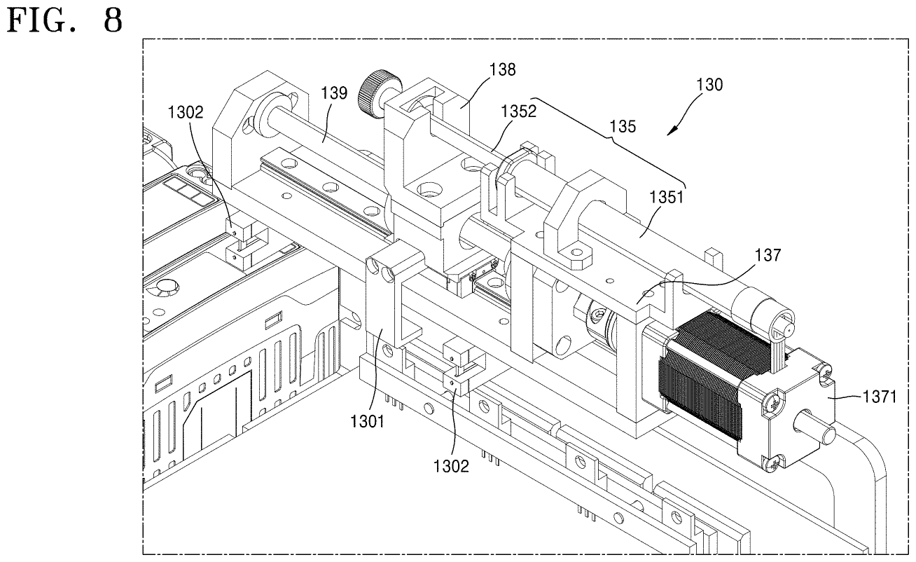

As illustrated in FIG. 8, the syringe pump 135 utilized in the embodiment includes a body part 1351 and a rod part 1352. The body part 1351 is fixed to a fixing part 137, and a guide block 138 is coupled to the rod part 1352. The guide block 138 is inserted over and supported by a second screw shaft 139 which is rotated by a second step motor 1371 coupled to the body part 1351. The second screw shaft 139 is rotated by the second step motor 1371 in the forward and reverse directions, and the guide block 138 is moved forward and backward by the rotation of the second screw shaft 139 to push or pull the rod part 1352. The connection pipe 131 is connected to a front end of the body part 1351, and accordingly, the suction force and ejection force are provided to the ejecting unit 10. The second screw shaft 139 is precisely machined similar to the first screw shaft 153 and may finely control the movement of the guide block 138.

Also, the guide block 138 is controlled to move within an appropriate range by a second probe 1301 provided in the guide block 138 and a second object detecting sensor 1302 for sensing the second probe 1301. A pair of second object detecting sensors 1302 are spaced a predetermined distance from each other, and when the second probe 1301 is sensed by the second object detecting sensors 1302 while moving forward and backward, the movement of the guide block 138 is limited.

[symbol]

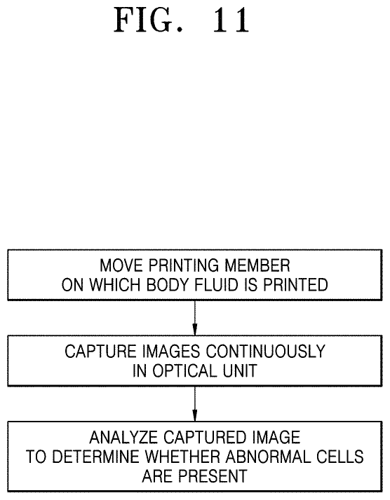

As described above, the body fluid analyzing apparatus according to the embodiment of the present invention moves a printing member 30 in one direction and prints a body fluid on the printing member 30 by ejecting the body fluid through a needle part 11 of the ejecting unit 10 as illustrated in FIG. 11. A single layer of blood cells is printed on the printing member 30 through the needle part 11, and the analysis of body fluid may be performed by continuously capturing and analyzing images of lots of blood cells. That is, the needle part 11 allows the body fluid to be printed as a single layer on the printing member 30 so that the blood cells constituting the body fluid do not overlap each other in the vertical direction, and the optical unit 70 continuously captures and analyses images of the printed body fluid. Therefore, steps of analyzing the body fluid may be simplified, and the body fluid may be rapidly and accurately analyzed.

According to the embodiment, the cartridge 60 may accommodate a printing member 30 having up to approximately 250 m, and the printing is performed while the printing member 30 is moved in one direction from the first rolling unit 40 to the second rolling unit 50. Thus, a significantly larger amount of body fluid may be printed compared to printing on slide glass in the related art, and there is an effect that all of the body fluid to be sampled may be analyzed because the images of the body fluid over all the areas printed on the printing member 30 are secured.

In the body fluid analyzing apparatus according to the embodiment of the present invention, the film-type printing member 30 is accommodated in the cartridge 60 to facilitate replacement and storage of the printing member 30. Also, the printing member 30 is moved in both directions within the cartridge 60, the printing is performed when moved in one direction, and the optical analysis is performed when moved in the other direction. Thus, the space of equipment may be effectively used and reduced.

[symbol]

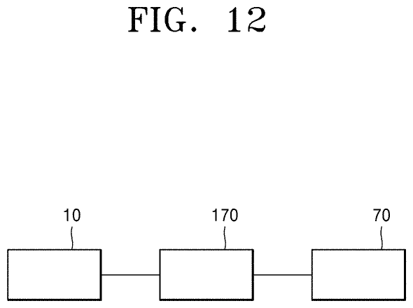

Also, the body fluid analyzing apparatus according to another embodiment of the present invention may include a staining unit 170 for selectively staining cells of the printed body fluid, as illustrated in FIG. 12. The staining unit 170 is provided to selectively stain blood cells, cancer cells, endothelial cells, epithelial cells, bacteria, parasites, cultured cells, exosomes, or the like, which are to be detected from the body fluid to be examined such as blood, lymph, tissue fluid, cerebrospinal fluid, in-vitro culture, chopped tissue, tears, amniotic fluid, or semen. After the body fluid is stained by the staining unit 170, an image of the stained body fluid is captured and analyzed by the optical unit 70. The image capturing and analysis may be performed in substantially the same manner as the process performed by the optical unit 70, and the image analysis may be more easily performed because targets to be detected are easily identified by the staining. Obviously, the embodiment provides the same configurations and operations as those according to the one embodiment described above, and thus, detailed descriptions thereof will be omitted.

[symbol]

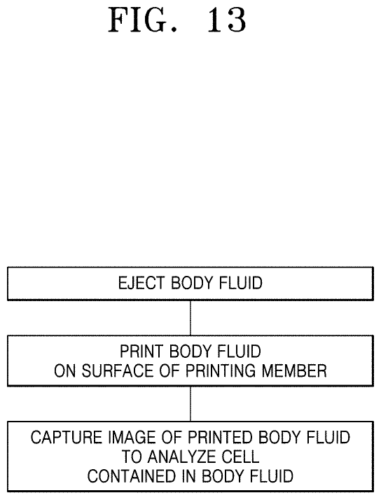

According to another aspect of the present invention, there is provided a body fluid analyzing method as illustrated in FIG. 13.

The body fluid analyzing method according to an embodiment of the present invention includes an ejecting step, a printing step, and an image capturing and analyzing step. The ejecting step is an ejection step of ejecting a body fluid, and the printing step is a step of, while moving a printing member 30 in one direction, printing on a surface of the printing member 30 when the body fluid is ejected in the ejecting step. Also, the image capturing and analyzing step is a step of capturing an image of the body fluid printed in the printing step and analyzing the body fluid.

In the embodiment, the body fluid is a biological sample separated from a subject, and the subject includes a human and an animal. Particularly, the body fluid includes blood, lymph, tissue fluid, cerebrospinal fluid, in-vitro culture, chopped tissue, tears, amniotic fluid, semen, urine, or the like. Also, during the image capturing and analyzing step, at least one of blood cells, cancer cells, endothelial cells, epithelial cells, bacteria, parasites, cultured cells, and exosomes, which are contained in the body fluid, may be analyzed.

In the embodiment, the ejecting step, the printing step, and the image capturing and analyzing step may be performed by a process in which, in the body fluid analyzing apparatus described above, a body fluid is printed by an ejecting unit 10 when a printing member 30 moves in one direction, and an image of the printed body fluid is captured and analyzed by an optical unit 70. Also, the body fluid analyzing method may include substantially the same process as that provided by the configuration of the body fluid analyzing apparatus described above, for example, a process or operation in which a body fluid is quantitatively ejected through a needle part 11 by a pressure supply unit 130.

In the embodiment, the printing member 30 may include a tape-type film. The printing member 30, which is wound into a roll shape on a first rolling unit 40 provided on one side, may be moved while being unwound from the first rolling unit 40 and be wound into a roll shape on a second rolling unit 50 provided on the other side. Also, the printing member 30, which is wound on the second rolling unit 50, may be moved while being unwound from the second rolling unit 50 and be wound again on the first rolling unit 40. Here, the printing step may be performed when the printing member 30 is moved to the second rolling unit 50 while being unwound from the first rolling unit 40, and the image capturing and analyzing step may be performed when the printing member 30 is moved to the first rolling unit 40 and wound again thereon while being unwound from the second rolling unit 50. As in the body fluid analyzing apparatus, the printing member 30 in the embodiment may be accommodated in a cartridge 60 in which the first rolling unit 40 and the second rolling unit 50 are provided, and be wound on or unwound from the first rolling unit 40 or the second rolling unit 50 while rotating in the forward and reverse directions.

In another embodiment, the first rolling unit 40 and the second rolling unit 50 are provided separate from each other, and the printing member may be wound on the second rolling unit 50 while being unwound from the first rolling unit 40 (not wound thereon again). Alternatively, the first rolling unit 40 and the second rolling unit 50 are provided separate from each other, and the printing member may be wound on the second rolling unit 50 while being unwound from the first rolling unit 40 and may be then wound again on the first rolling unit 40 while being unwound from the second rolling unit 50.

Also, the printing step and the image capturing and analyzing step may be performed continuously while the printing member 30 moves in one direction. For example, before the printing member is wound on the second rolling unit 50 while being unwound from the first rolling unit 40, the printing step and the image capturing and analyzing step may be performed. Obviously, in the case where the printing member 30 is not wound again, the printing of the body fluid is performed on the printing member 30, and then, the image capturing and analyzing step may be performed.

[symbol]

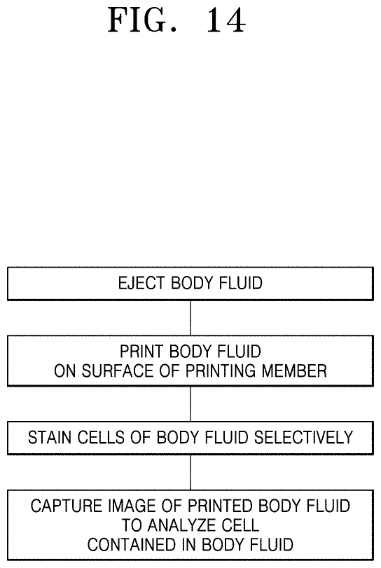

Also, a body fluid analyzing method according to another embodiment of the present invention as illustrated in FIG. 14 may include a staining step in addition to the steps according to the embodiment of FIG. 13.

The staining step is provided to selectively stain cells of the printed body fluid. The staining step is provided to selectively stain targets to be detected from the body fluid to be examined. The staining step is provided to selectively stain the targets to be detected after the body fluid is printed on the printing member 30, and the image capturing and analyzing step is to capture and analyze an image of the body fluid having the stained cells. The image capturing and analyzing step may be performed in substantially the same manner as the process performed by the optical unit in the body fluid analyzing apparatus, and the image analysis may be more easily performed because the targets to be detected are easily identified by the staining. Obviously, the embodiment provides the same configurations and operations as those according to the one embodiment described above, and thus, detailed descriptions thereof will be omitted.

[symbol]

Although the exemplary embodiments of the present invention have been described in detail, the present invention is not be limited to these exemplary embodiments, but various modifications can be provided within the scope of the present invention.

### EXPLANATION OF REFERENCE NUMERALS DESIGNATING THE MAJOR ELEMENTS OF THE DRAWINGS

10 . . . ejecting unit 11 . . . needle part
12 . . . mounting part 121 . . . upper plate
122 . . . lower plate 123 . . . exposure hole
13 . . . height adjusting part 20 . . . main body unit
21 . . . first rotary shaft 22 . . . second rotary shaft
23 . . . coupling pin 24 . . . mounting bracket
30 . . . printing member 40 . . . first rolling unit
41 . . . first hook part 50 . . . second rolling unit
51 . . . second hook part 60 . . . cartridge
61 . . . first exposure part 62 . . . second exposure part
63 . . . first insertion hole 64 . . . second insertion hole
65 . . . cartridge front part 66 . . . cartridge rear part
67 . . . first support piece 68 . . . second support piece
69 . . . coupling protrusion 651 . . . elongated hole
652 . . . coupling hole 691 . . . pin hole
692 . . . guide roller 70 . . . optical unit
71 . . . light source part 72 . . . image capturing part
73 . . . image analyzing part 74 . . . light collecting part
721 . . . lens part 80 . . . power providing unit
81 . . . first clutch 82 . . . second clutch
83 . . . connection member 90 . . . first tension unit
91 . . . first belt 100 . . . second tension unit
101 . . . second belt 110 . . . speed measuring unit
1101 . . . shaft 120 . . . sensing unit
1201 . . . pressure sensor 1202 . . . pin part
130 . . . pressure supply unit 131 . . . connection pipe
132 . . . conveyance pipe 133 . . . branch pipe
134 . . . valve 135 . . . syringe pump
1351 . . . body part 1352 . . . rod part
136 . . . control part 137 . . . fixing part
138 . . . guide block 139 . . . second screw shaft
1301 . . . second probe 1302 . . . second object detecting sensor
1303 . . . oil tank 1371 . . . second step motor
140 . . . power supply unit 151 . . . support block
152 . . . moving block 153 . . . first screw shaft
154 . . . support part 155 . . . first step motor
160 . . . loading block 161 . . . slot
162 . . . loading block driving part 163 . . . first probe
164 . . . first object detecting sensor 170 . . . staining unit

1. A body fluid analyzing apparatus comprising:
an ejecting unit configured to eject a body fluid;
a main body unit configured to support an ejecting unit; and
a printing member on which the body fluid ejected through the ejecting unit is printed,
wherein the body fluid is printed on a surface of the printing member while the printing member moves in one direction relative to the ejecting unit.

2. The body fluid analyzing apparatus of claim 1,
wherein the printing member, which is wound into a roll shape on a first rolling unit provided on one side, is moved while being unwound from the first rolling unit and is wound again into a roll shape on a second rolling unit provided on the other side, and
the body fluid ejected from the ejecting unit is printed on the printing member when the printing member is moved to the second rolling unit while being unwound from the first rolling unit and is wound in a printed state on the second rolling unit.

3. (canceled)

4. The body fluid analyzing apparatus of claim 1,
the printing member is accommodated in a cartridge that is detachably coupled to the main body unit,
wherein a first rolling unit and a second rolling unit, which are coupled to respective ends of the printing member, are provided in the cartridge, and
the printing member is wound on the second rolling unit while being unwound from the first rolling unit, or is wound on the first rolling unit while being unwound from the second rolling unit.

5. (canceled)

6. The body fluid analyzing apparatus of claim 1,
wherein the printing member is a film comprising a tape-type light transmitting material.

7. The body fluid analyzing apparatus of claim 2,
comprising an optical unit which is provided in the main body unit to capture and analyze an image of the body fluid printed on the printing member,
wherein, when the printing member is moved to the first rolling unit while being unwound from the second rolling unit and is wound again on the first rolling unit, the optical unit captures and analyzes the image of the body fluid printed on the printing member.

8. The body fluid analyzing apparatus of claim 7,
wherein the optical unit comprises:
a light source part which is coupled to the main body unit to provide a light source toward the printing member;
an image capturing part which is positioned on the opposite side from the light source part to capture the image of the body fluid printed on the printing member; and
an image analyzing part which uses the image captured by the image capturing part to perform image analysis.

9. The body fluid analyzing apparatus of claim 2, comprising:
a first rotary shaft and a second rotary shaft which are provided in the main body unit and inserted into the first rolling unit and the second rolling unit, respectively;
a first clutch selectively connected to the first rotary shaft;
a second clutch selectively connected to the second rotary shaft;
a power providing unit configured to transmit power to the first clutch or the second clutch; and
a connection member configured to connect the first clutch to the second clutch and transmit the power,
wherein the first clutch or the second clutch is selectively connected to the first rotary shaft or the second rotary shaft, respectively, by the power providing unit to selectively rotate the first rolling unit or the second rolling unit, thereby moving the printing member in one direction and winding the printing member.

10. (canceled)

11. The body fluid analyzing apparatus of claim 2,
comprising a speed measuring unit configured to sense a moving speed of the printing member when the printing member is unwound from the first rolling unit or the second rolling unit.

12. The body fluid analyzing apparatus of claim 1,
wherein the ejecting unit comprises:
a needle part configured to eject the body fluid;
a mounting part to which the needle part is mounted; and
a height adjusting part configured to space an end of the needle part from the printing member a predetermined distance.

13. The body fluid analyzing apparatus of claim 12,
wherein the height adjusting part comprises a sensing unit configured to sense whether or not the end of the needle part comes into contact with the printing member when the mounting part approaches the printing member.

14. The body fluid analyzing apparatus of claim 12,
wherein the needle part comprises a chamber made of a transmitting material so that the body fluid is visible from the outside.

15. (canceled)

16. The body fluid analyzing apparatus of claim 1,
a pressure supply unit which provides pressure so that the body fluid to be printed is suctioned into the electing unit or the body fluid is elected from the electing unit,
wherein the pressure supply unit comprises:
a connection pipe connected to the ejecting unit;
a conveyance pipe configured to convey oil from an oil tank which is filled with the oil;
a branch pipe branched from an intersection point between the connection pipe and the conveyance pipe;
a valve configured to selectively open and close the connection pipe, the conveyance pipe, and the branch pipe;
a syringe pump which provides pressure so that the oil is suctioned through the conveyance pipe when the valve allows the conveyance pipe to communicate with the branch pipe, and the needle part suctions or ejects the body fluid when the valve allows the connection pipe to communicate with the branch pipe; and
a control part configured to control an output of the syringe pump.

17. The body fluid analyzing apparatus of claim 4,
wherein the cartridge comprises:
a first exposure part which is exposed to the outside so that the body fluid is printed when the printing member is moved to the second rolling unit while being unwound from the first rolling unit; and
a second exposure part which is exposed to the outside so that an image of the printed body fluid is captured when the printing member wound on the second rolling unit is moved to the first rolling unit while being unwound from the second rolling unit again,
wherein a first insertion hole, into which a light source part that provides a light source to capture the image of the printed body fluid is inserted, is provided below the second exposure part.

18. The body fluid analyzing apparatus of claim 1,
wherein the body fluid comprises blood, lymph, tissue fluid, cerebrospinal fluid, in-vitro culture, chopped tissue, tears, amniotic fluid, semen, or urine.

19. The body fluid analyzing apparatus of claim 7,
wherein the optical unit analyzes at least one of blood cells, cancer cells, endothelial cells, epithelial cells, bacteria, parasites, cultured cells, and exosomes, which are contained in the body fluid.

20. (canceled)

21. A body fluid analyzing method comprising:
an ejecting step of ejecting body fluid from an ejecting unit;
a printing step of, while moving a printing member in one direction, printing the body fluid on a surface of the printing member when the body fluid is ejected by the ejecting step; and
an image capturing and analyzing step of capturing an image of the body fluid printed by the printing step and analyzing the body fluid.

22. The body fluid analyzing method of claim 21,
wherein the printing member comprises a tape-type film,
wherein the printing member, which is wound into a roll shape on a first rolling unit provided on one side, is moved while being unwound from the first rolling unit and is wound into a roll shape on a second rolling unit provided on the other side, and the printing member, which is wound on the second rolling unit, is moved while being unwound from the second rolling unit and is wound again on the first rolling unit,
wherein the printing step is performed when the printing member is moved to the second rolling unit while being unwound from the first rolling unit, and
the image capturing and analyzing step is performed when the printing member is moved to the first rolling unit while being unwound from the second rolling unit and is wound again on the first rolling unit.

23. The body fluid analyzing method of claim 21,
wherein the body fluid comprises blood, lymph, tissue fluid, cerebrospinal fluid, in-vitro culture, chopped tissue, tears, amniotic fluid, semen, or urine.

24. The body fluid analyzing method of claim 21,
wherein in the image capturing and analyzing step, at least one of blood cells, cancer cells, endothelial cells, epithelial cells, bacteria, parasites, cultured cells, and exosomes, which are contained in the body fluid, are analyzed.

25. The body fluid analyzing method of claim 21,
comprising a staining step of selectively staining cells of the printed body fluid,
wherein, in the image capturing and analyzing step, an image of the body fluid having the printed cells is captured and analyzed.
